(19) United States
(12) Patent Application Publication
(10) Pub. No.: US 2004/0073967 A1
(43) Pub. Date: Apr. 15, 2004

(54) Nucleic acid intergration in eukaryotes

(75) Inventors: Paul Jan, J. Hooykaas, Oegstgeest (NL); Haico van Attikum, Leiden (NL); Paul Bundock, Amsterdam (NL)

Correspondence Address: TRASK BRITT, P.O. BOX 2550, SALT LAKE CITY, UT, 84110, US

(73) Assignee: Universiteit Leiden, Leiden (NL)

(21) Appl. No.: 10/601,084

(22) Filed: Jun. 20, 2003

Related U.S. Application Data
(63) Continuation of application No. PCT/NL01/00936, filed on Dec. 21, 2001.

(30) Foreign Application Priority Data
Dec. 22, 2000 (EP) 00204693.6

(51) Int. Cl. A01H 1/00; C12N 15/82

(52) U.S. Cl. 800/278; 435/468

(57) ABSTRACT

The invention relates to methods for directing integration of a nucleic acid of interest towards homologous recombination and uses thereof. The present invention discloses factors involved in integration of a nucleic acid by illegitimate recombination which provides a method of directing integration of a nucleic acid of interest to a predetermined site, whereby the nucleic acid has a homology at or around the predetermined site, in a eukaryote with a preference for non-homologous recombination comprising steering an integration pathway towards homologous recombination. Furthermore, the invention provides a method of directing integration of a nucleic acid of interest to a subtelomeric and/or telomeric region in a eukaryote with a preference for non-homologous recombination.

### CROSS-REFERENCE TO RELATED APPLICATION

This application is a continuation of PCT International Patent Application No. PCT/NL/01/00936, filed on Dec. 21, 2001, designating the United States of America, and published, in English, as PCT International Publication No. WO 02/052026 on Jul. 4, 2002, the contents of the entirety of which is incorporated by this reference.

### TECHNICAL FIELD

The invention relates generally to the field of molecular biology and cell biology. It particularly relates to methods to direct integration towards homologous recombination and uses thereof.

### BACKGROUND

Several methods are known to transfer nucleic acids to, in particular, eukaryotic cells. In some methods, the nucleic acid of interest is transferred to the cytoplasm of the cell; in some, the nucleic acid of interest is integrated into the genome of the host. Many different vehicles for transfer of the nucleic acid are known. For different kinds of cells, different systems can be used, although many systems are more widely applicable than just a certain kind of cells. In plants, e.g., a system based on Agrobacterium tumefaciens is often applied. This system is one of the systems that are used in a method according to the invention.

State of the Art: The soil bacterium Agrobacterium tumefaciens is able to transfer part of its tumor-inducing (Ti) plasmid, the transferred (T-) DNA, to plant cells. This results in crown gall tumor formation on plants due to expression of onc-genes, which are present on the T-DNA. Virulence (vir) genes, located elsewhere on the Ti-plasmid, mediate T-DNA transfer to the plant cell. Some Vir proteins accompany the T-DNA during its transfer to the plant cell to protect the T-DNA and to mediate its transfer to the plant nucleus. Once in the plant nucleus, the T-DNA is integrated at a random position into the plant genome (reviewed by Hooykaas and Beijersbergen, 1994, and Hansen and Chilton, 1999). Removal of the onc-genes from the T-DNA does not inactivate T-DNA transfer. T-DNA, disarmed in this way, is now the preferred vector for the genetic modification of plants.

Although much is known about the transformation process, not much is known about the process by which the T-DNA is integrated into the plant genome. It is likely that plant enzymes mediate this step of the transformation process (Bundock et al., 1995). The integration pattern of T-DNA in transformed plants has been extensively studied (Matsumoto et al., 1990; Gheysen et al., 1991; Meyerhofer et al., 1991). The results indicated that T-DNA integrates via illegitimate recombination (IR) (also called nonhomologous recombination; both terms may be used interchangeably herein), a process which can join two DNA molecules that share little or no homology (here the T-DNA and plant target DNA). Even T-DNA molecules in which a large segment of homologous plant DNA was present integrated mainly by IR and only with very low frequency (1:10⁴−10⁵ ) by homologous recombination (HR) (Offringa et al., 1990).

Recently, it was shown that Agrobacterium, is not only able to transfer its T-DNA to plant cells, but also to other eukaryotes, including the yeast S. cerevisiae (Bundock et al., 1995) and a wide variety of filamentous fungi (de Groot et al., 1998). In S. cerevisiae, T-DNA carrying homology with the yeast genome integrates via HR (Bundock et al., 1995). However, T-DNA lacking any homology with the S. cerevisiae genome becomes integrated at random positions in the genome by the same IR process as is used in plants (Bundock and Hooykaas, 1996). Apparently, eukaryotic cells have at least two separate pathways (one via homologous recombination and one via nonhomologous recombination) through which nucleic acids (in particular, of course, DNA) can be integrated into the host genome. The site of integration into a host cell genome is important with respect to the likelihood of transcription and/or expression of the integrated nucleic acid. The present invention provides methods and means to direct nucleic acid integration to a predetermined site through steering integration towards the homologous recombination pathway. The present invention arrives at such steering either by enhancing the HR pathway or by inhibiting (meaning reducing) the IR pathway.

Host factors involved in the integration of nucleic acid by IR have not so far been identified. The present invention discloses such factors which enables the design of methods for their (temporary) inhibition, so that integration of nucleic acid by IR is prevented or more preferably completely inhibited, shifting the integration process towards HR and facilitating the isolation of a host cell with nucleic acid integrated by HR at a predetermined site. This is extremely important, since there is no method available yet for easy and precise genetic modification of a host cell using HR (gene targeting). Of course, the actual site of integration is then determined by homology of the nucleic acid of interest with the site.

### BRIEF SUMMARY OF THE INVENTION

In a first embodiment, the invention provides a method of directing integration of a nucleic acid of interest to a predetermined site, whereby the nucleic acid has homology at or around the predetermined site, in a eukaryote with a preference for nonhomologous recombination comprising steering an integration pathway towards homologous recombination. Preferably, such a method comprises at least the steps of introducing the nucleic acid of interest to a cell of the eukaryote, for example, by the process of transformation or electroporation, and integration of the nucleic acid in the genetic material of the cell. Integration is a complex process wherein a nucleic acid sequence becomes part of the genetic material of a host cell. One step in the process of nucleic acid integration is recombination; via recombination, nucleic acid sequences are exchanged or inserted and the introduced nucleic acid becomes part of the genetic material of a host cell. In principle, two different ways of recombination are possible: homologous and illegitimate or nonhomologous recombination. Most (higher) eukaryotes do not, or at least not significantly, practice homologous recombination, although the essential proteins to accomplish such a process are available. One reason for this phenomenon is that frequent use of homologous recombination in (higher) eukaryotes could lead to undesirable chromosomal rearrangements due to the presence of repetitive nucleic acid sequences. To accomplish homologous recombination via a method according to the invention, it is important to provide a nucleic acid which has homology with a predetermined site. It is clear to a person skilled in the art that the percentage of homology and the length of homologous regions play an important role in the process of homologous recombination. The percentage of homology is preferably close to 100%. A person skilled in the art is aware of the fact that lower percentages of homology are also used in the field of homologous recombination but dependent on, for example, the regions of homology and their overall distribution, which can lead to a lower efficiency of homologous recombination but are still useful and, therefore, included in the present invention. Furthermore, the length of a nearly homologous region is approximately 3 kb, which is sufficient to direct homologous recombination. At least one homologous region is necessary for recombination but, more preferably, two homologous regions flanking the nucleic acid of interest are used for targeted integration. The researcher skilled in the art knows how to select the proper percentage of homology, the length of homology and the amount of homologous regions. By providing such a homology, a nucleic acid is integrated at every desired position within the genetic material of a host cell. It is clear to a person skilled in the art that the invention as disclosed herein is used to direct any nucleic acid (preferably DNA) to any predetermined site as long as the length of homology and percentage of homology are high enough to provide/enable homologous recombination. A predetermined site is herein defined as a site within the genetic material contained by a host cell to which a nucleic acid with homology to this same site is integrated with a method according to the invention. It was not until the present invention that a nucleic acid is integrated at every desired position and, therefore, a method according to the invention is applied, for example, to affect the gene function in various ways, not only for complete inactivation but also to mediate changes in the expression level or in the regulation of expression, changes in protein activity or the subcellular targeting of an encoded protein. Complete inactivation, which usually cannot be accomplished by existing methods such as antisense technology or RNAi technology (Zrenner et al., 1993), is useful, for instance, for the inactivation of genes controlling undesired side branches of metabolic pathways, for instance, to increase the quality of bulk products such as starch, or to increase the production of specific secondary metabolites or to inhibit formation of unwanted metabolites. A method according to the invention is also used to inactivate genes controlling senescence in fruits and flowers or that determine flower pigments. Replacement of existing regulatory sequences by alternative regulatory sequences is used to alter expression of in situ modified genes to meet requirements (e.g., expression in response to particular physical conditions such as light, drought or pathogen infection, or in response to chemical inducers, or depending on the developmental state (e.g., in a storage organ, or in fruits or seeds) or on tissue or cell types). Also, a method according to the invention is used to effectuate predictable expression of transgenes encoding novel products, for example, by replacing existing coding sequences of genes giving a desired expression profile by those for a desired novel product. For example, to produce proteins of medicinal or industrial value in the seeds of plants, the coding sequence of a strongly expressed storage protein may be replaced by that of the desired protein. As another example, existing coding sequences are modified so that the encoded protein has optimized characteristics, for instance, to make a plant herbicide tolerant, to produce storage proteins with enhanced nutritional value, or to target a protein of interest to an organelle or to secrete it to the extracellular space. As yet another example, eukaryotic cells (including yeast, fungus, plant, mammalian cells or nonhuman animal cells) are provided with a gene encoding a protein of interest integrated into the genome at a site which ensures high expression levels. As another example, the nucleic acid of interest can be part of a gene delivery vehicle to deliver a gene of interest to a eukaryotic cell in vitro or in vivo. In this way, a defective p53 can be replaced by an intact p53. In this way, a tumoricidal gene is delivered to a predetermined site present only in, e.g., proliferating cells, or present only in tumor cells, for example, to the site from which a tumor antigen is expressed. Gene delivery vehicles are well known in the art and include adenoviral vehicles, retroviral vehicles, nonviral vehicles such as liposomes, etc. As another example, the invention is used to produce transgenic organisms. Knockout transgenics are already produced by homologous recombination methods. The present invention improves the efficiency of such methods. Also, transgenics with desired properties are made. It is clear to a person skilled in the art that transgenics can, for example, be made by the use of Agrobacterium as a gene delivery vehicle for plant (Vergunst et al., 1998), yeast (Bundock et al., 1995), fungus (de Groot et al., 1998) or animal (Kunik et al., 2001) or by direct DNA delivery methods exemplified by, but not restricted to, electroporation for yeast (Gietz & Woods, 2001), plant (D'Halluin et al., 1992; Lin et al., 1997), fungus (Ozeki et al., 1994) and animal (Templeton et al., 1997), LiCl treatment for yeast (Schiestl et al., 1993), microinjection for plant (Schnorf et al., 1991) and animal (Capecchi, 1980) and “DNA whiskers” for plant (Kaeppler et al., 1990; Dunwell, 1999) or particle bombardment for plants and animals (Klein et al., 1992). It is, furthermore, clear that transgenic plants can be obtained via selective regeneration of transformed plant cells into a complete fertile plant (Vergunst et al., 1998) or via nonregenerative approaches by transforming germ line cells exemplified by, but not restricted to, dipping Arabidopsis flowers into an Agrobacterium suspension (Bechtold et al., 1993). It is also clear that transgenic animals can be obtained by transforming embryonic stem cells with one of the DNA delivery methods mentioned above (Hooper, 1992).

In another embodiment, the invention provides a method of directing nucleic acid integration to a predetermined site, whereby the nucleic acid has homology at or around the predetermined site, in a eukaryote with a preference for nonhomologous recombination comprising steering an integration pathway towards homologous recombination by providing a mutant of a component involved in nonhomologous recombination. Methods to identify components involved in nonhomologous recombination are outlined in the present description wherein S. cerevisiae was used as a model system. To this end, several yeast derivatives defective for genes known to be involved in various recombination processes were constructed and the effect of the mutations on T-DNA integration by either HR or IR was tested. The results as disclosed herein show that the proteins encoded by YKU70, RAD50, MRE11, XRS2, LIG4 and SIR4 play an essential role in DNA integration by IR but not by HR. WO 00/12716 describes a maize Ku70 orthologue and suggests that “Control of homologous recombination or nonhomologous end joining by modulating Ku provides the means to modulate the efficiency [sic, with] which heterologous nucleic acids are incorporated into the genomes of a target plant cell.” WO 00/68404 describes a maize Rad50 orthologue and suggests an analogous control for Rad50. Both patent applications, however, do not disclose, in contrast to the present patent application, that by preventing or more preferably completely inhibiting nonhomologous recombination, for example, by providing a mutant of a component involved in nonhomologous recombination or by inhibiting such a component, the integration pathway is steered towards homologous recombination. It is clear to a person skilled in the art that different mutants of a component involved in nonhomologous recombination exist. Examples are deletion mutants, knockout (for example, via insertion) mutants or point mutants. Irrespective of the kind of mutant, it is important that a component involved in nonhomologous recombination is no longer capable or at least significantly less capable to perform its function in the process of nonhomologous recombination. As disclosed herein, disruption of YKU70, RAD50, MREI11, XRS2, LIG4 and SIR4 did not affect the frequency of DNA integration by HR, showing that these genes are not involved in DNA integration by HR, but only in DNA integration by IR. Moreover, in the wild-type yeast strain, 85% of the integration events occurred by HR (37% by replacement and 63% by insertion) and 15% by IR. In contrast, integration occurred only by HR in yeast strains lacking ku70 or lig4. In rad50 and xrs2 mutant strains, the T-DNA preferentially integrated by HR (92%) and 93% of these T-DNAs integrated by replacement and only 7% by insertion. Thus, the absence of a functional rad50 or xrs2 gene leads to a significantly increased frequency of replacement reactions.

In another embodiment, the invention provides a method of directing integration of a nucleic acid of interest to a subtelomeric and/or telomeric region in a eukaryote with a preference for nonhomologous recombination by providing a mutant of a component involved in nonhomologous recombination. A telomeric region is typically defined as a region containing repetitive sequences which is located at the end of a chromosome. A subtelomeric region is typically defined as a region flanking the telomeric region. As an example, it is disclosed herein that in yeast strains carrying disruptions of RAD50, MRE11 or XRS2, the distribution of integrated DNA copies is altered when compared to wild-type. DNA becomes preferentially integrated in telomeres or subtelomeric regions in the rad50, mre11 and xrs2 mutants. A great advantage of integration of DNA copies in telomeres or subtelomeric regions instead of integration elsewhere in the genomic material is that there is no danger for host genes being mutated or inactivated by a DNA insertion. When in plants deficient for RAD50, MRE11 or XRS2, DNA copies also integrate into telomeres or subtelomeric regions. Such plants are used for subtelomeric targeting of T-DNA in transformation experiments to prevent additional insertion mutations from random T-DNA integration into the plant genome.

In yet another embodiment, the invention provides a method of directing nucleic acid integration to a predetermined site, whereby the nucleic acid has homology at or around the predetermined site, in a eukaryote with a preference for nonhomologous recombination comprising steering an integration pathway towards homologous recombination by partially or more preferably completely inhibiting a component involved in nonhomologous recombination. Partial or complete inhibition of a component involved in nonhomologous recombination is obtained by different methods, for example, by an antibody directed against such a component or a chemical inhibitor or a protein inhibitor or peptide inhibitor or an antisense molecule or an RNAi molecule. Irrespective of the kind of (partial or more preferably complete) inhibition, it is important that a component involved in nonhomologous recombination is no longer capable or at least significantly less capable to perform its function in the process of nonhomologous recombination. In yet another embodiment, the invention provides a method of directing integration of a nucleic acid of interest to a subtelomeric and/or telomeric region in a eukaryote with a preference for nonhomologous recombination by partially or more preferably completely inhibiting a component involved in nonhomologous recombination. Preferably, the component involved in nonhomologous recombination is rad50, mre11 or xrs2.

In a preferred embodiment, the invention provides a method of directing nucleic acid integration to a predetermined site or to a subtelomeric and/or telomeric region by providing a mutant of a component involved in nonhomologous recombination or by partially or more preferably completely inhibiting a component involved in nonhomologous recombination wherein the component comprises ku70, rad50, mre11, xrs2, lig4, sir4 or others such as ku80 (Tacciole et al., 1994; Milne et al., 1996), lif1 (Teo and Jackson, 2000; XRCC4 in human, see FIG. 6; Junop et al., 2000) and nej1, (Kegel et al., 2001; Valencia et al., 2001). Components involved in nonhomologous recombination are identified as outlined in the present description. The nomenclature for genes as used above is specific for yeast. Because the nomenclature of genes differs between organisms, a functional equivalent or a functional homologue (for example, NBS1, a human xrs2 equivalent (Paull and Gellert, 1999) and see, for example, FIGS. 2 to 5) and/or a functional fragment thereof, all defined herein as being capable of performing (in function, not in amount) at least one function of the yeast genes ku70, rad50, mre11, xrs2, lig4, sir4, ku80, lif1 or nej1, are also included in the present invention. A mutant of a component directly associating with a component involved in nonhomologous recombination or partial or complete inhibition of a component directly associating with a component involved in nonhomologous recombination is also part of this invention. Such a component directly associating with a component involved in nonhomologous recombination is, for example, identified in a yeast two-hybrid screening. An example of a component directly associating with a component involved in nonhomologous recombination is KU80, which forms a complex with KU70. In a more preferred embodiment, the invention provides a method of directing nucleic acid integration in yeast, fungus, plant or nonhuman animal cells.

In another embodiment, the invention provides a method of directing nucleic acid integration to a predetermined site, whereby the nucleic acid has homology at or around the predetermined site, in a eukaryote with a preference for nonhomologous recombination comprising steering an integration pathway towards homologous recombination by transiently (partially or more preferably completely) inhibiting integration via nonhomologous recombination. In yet another embodiment, the invention provides a method of directing integration of a nucleic acid of interest to a subtelomeric and/or telomeric region in a eukaryote with a preference for nonhomologous recombination by transiently (partially or more preferably completely) inhibiting integration via nonhomologous recombination. In a more preferred embodiment, such a method is used for yeast, plant, fungus or nonhuman animal and the transient (partial or more preferably complete) inhibition is provided by a preferably stably inserted and expressed chimeric transgene that encodes a peptide inhibitory to one, some or all nonhomologous recombination (NHR) enzymes fused to a nuclear localization signal (Hanover, 1992; Raikhel, 1992) and the steroid-binding domain of a steroid receptor (Picard et al., 1988). The chimeric transgene is constructed in such a way, using either heterologous or nonheterologous promoter sequences and other expression signals, that it provides stable expression in the target cells or tissue for transformation. In the absence of the steroid hormone, the steroid-binding domain binds to chaperone proteins, and thereby the fusion protein is retained in the cytoplasm. Upon treatment with the steroid hormone, the chaperones are released from the steroid-binding domain and the inhibitory peptide will enter the nucleus where it will interact with and inhibit the action of NHR enzymes. An example of an inhibitory peptide is a KU80 fragment that imparts radiosensitivity to Chinese hamster ovary cells (Marangoni et al., 2000).

In a more preferred embodiment, such a method is used for yeast, plant, fungus or a nonhuman animal and the transient (partial or more preferable complete) inhibition is provided by an Agrobacterium Vir-fusion protein capable of (partially or more preferably completely) inhibiting a component involved in nonhomologous recombination or capable of (partially or more preferably completely) inhibiting a functional equivalent or homologue thereof or capable of (partially or more preferably completely) inhibiting a component directly associating with a component involved in nonhomologous recombination. In an even more preferred embodiment, such an Agrobacterium Vir-fusion protein comprises VirF or VirE2. It was shown that the Agrobacterium VirF and VirE2 proteins are directly transferred from Agrobacterium to plant cells during plant transformation (Vergunst et al., 2000). To, for example, accomplish T-DNA integration by HR in plants, VirF-fusion proteins containing, for example, a peptide inhibitor of IR in plant cells are introduced concomitantly with the targeting T-DNA. It has been reported that the C-terminal part (approximately 40 amino acids) of VirF or VirE2 is sufficient to accomplish transfer of T-DNA. A functional fragment and/or a functional equivalent of VirF or VirE is, therefore, also included in the present invention. Preferably, the nucleic acid of interest is delivered to a cell of the eukaryote by Agrobacterium.

In an even more preferred embodiment, a component involved in nonhomologous recombination comprises ku70, rad50, mre11, xrs2, lig4, sir4, ku80, lif1or nej1 or functional equivalents or homologue thereof or associating components. The nomenclature for genes as used above is specific for yeast. Because the nomenclature of genes differs between organisms, a functional equivalent or a functional homologue (see, for example, FIGS. 2 to 5) and/or a functional fragment thereof, all defined herein as being capable of performing (in function, not in amount) at least one function of the yeast genes ku70, rad50, mre11, xrs2, lig4, sir4, ku80, lif1or nej₁, are also included in the present invention. By transiently (partially or more preferably completely) inhibiting a component involved in nonhomologous recombination, a nucleic acid is integrated at any desired position without permanently modifying a component involved in nonhomologous recombination and preventing unwanted side effects caused by the permanent presence of such a modified component involved in nonhomologous recombination.

Methods according to the present invention, as extensively but not limiting discussed above, are used in a wide variety of applications. One embodiment of the present invention is the replacement of an active gene by an inactive gene according to a method of the invention. Complete inactivation, which usually cannot be accomplished by existing methods such as antisense technology or RNAi technology, is useful, for instance, for the inactivation of genes controlling undesired side branches of metabolic pathways, for instance, to increase the quality of bulk products such as starch, to increase the production of specific secondary metabolites or to inhibit formation of unwanted metabolites, and to inactivate genes controlling senescence in fruits and flowers or to determine flower pigments. Another embodiment of the present invention is the replacement of an inactive gene by an active gene. One example is the replacement of a defective p53 by an intact p53. Many tumors acquire a mutation in p53 during their development which renders it inactive and often correlates with a poor response to cancer therapy. By replacing the defect p53 by an intact p53, for example, via gene therapy, conventional anticancer therapy has a better chance of succeeding. In yet another embodiment of the invention, a therapeutic proteinaceous substance is integrated via a method of the invention. In this way, a tumoricidal gene is delivered to a predetermined site present only in e.g. proliferating cells, or present only in tumor cells, e.g., to the site from which a tumor antigen is expressed. In yet another embodiment, the invention provides a method to introduce a substance conferring resistance for an antibiotic substance to a cell. Also, a method according to the invention is used to confer a desired property to a eukaryotic cell. In a preferred embodiment, a gene delivery vehicle is used to deliver a desired nucleic acid to a predetermined site. Gene delivery vehicles are well known in the art and include adenoviral vehicles, retroviral vehicles, nonviral vehicles such as liposomes, etc. In this way, for example, a tumoricidal gene can be delivered to a predetermined site present only in, e.g., proliferating cells, or present only in tumor cells, e.g. to the site from which a tumor antigen is expressed.

Furthermore, a method according to the invention is used to improve gene-targeting efficiency. Such a method is used to improve, for example, the gene-targeting efficiency in plants. In plants, transgenes integrate randomly into the genome by IR (Mayerhof et al., 1991; Gheysen et al., 1991). The efficiency of integration by HR is very low, even when large stretches of homology between the transgene and the genomic target site are present (Offringa et al., 1990). Therefore, the efficiency of gene targeting using HR is very low in plants. The results that are disclosed herein show how to improve the gene-targeting efficiency in plants. From the fact that T-DNA integration by IR is strongly reduced in KU70-, RAD50-, MRE11-, XRS2-, LIG4- and SIR4-deficient yeast strains and T-DNA integration by HR is not affected in such strains, T-DNA integration by HR is more easily obtained in plants deficient for either of these genes. Recently, we have cloned a KU70 homologue of Arabidopsis thaliana (see FIG. 2, Bundock 2000, unpublished data). RAD50, MRE11 and LIG4 homologues have already been found in A. thaliana (GenBank accession numbers AF168748, AJ243822 and AF233527, respectively; see also FIGS. 3, 4 and 5 (Hartung and Puchta, 1999)). Currently, screenings are being performed to find plants carrying a T-DNA inserted in AtMRE11, AtKU70 or AtLIG4. These knockout plants are used to test whether T-DNA integration by IR is reduced and integration by HR is essentially unaffected, thereby facilitating the detection of T-DNA integration by HR.

Furthermore, the invention provides a method of directing integration of a nucleic acid of interest to a predetermined site, whereby the nucleic acid has homology at or around the predetermined site, in a eukaryote with a preference for nonhomologous recombination, comprising steering an integration pathway towards homologous recombination, wherein the nucleic acid sequence of interest is essentially replacing a sequence within the eukaryote. As disclosed herein within the experimental part, in the wild-type yeast strain, 85% of the integration events occurred by HR (37% by replacement and 63% by insertion) and 15% by IR. In contrast, integration occurred only by HR in yeast strains lacking ku70 or lig4. In rad50and xrs2 mutant strains, the T-DNA preferentially integrated by HR (92%) and 93% of these T-DNAs integrated by replacement and only 7% by insertion. Thus, the absence of a functional rad50or xrs2 gene leads to a significantly increased frequency of the desired replacement reactions.

The invention will be explained in more detail in the following description, which is not limiting to the invention.

### BRIEF DESCRIPTION OF DRAWINGS

FIG. 1: Junction sequences of T-DNA and S. cerevisiae genomic DNA. S. cerevisiae YPH250 (WT), rad50, mre11 and xrs2 strains were cocultivated with LBA111 9(pSDM8000). G418-resistant colonies were obtained. Chromosomal DNA was isolated and subjected to Vectorette PCR to determine the sequence of genomic DNA flanking the T-DNA. The position of T-DNA integration was determined by basic BLAST search of the yeast genome at http:/www.genome-stanford.edu/SGD. The Watson strand of genomic DNA that is fused to the LB or RB is shown in italics. Bold sequences represent sequence homology between the LB and target site. The filler DNA sequence is underlined and depicted in italics. The numbers above the LB sequences represent the number of bp deleted from the LB. Tel.=telomeric, Subtel.=subtelomeric and Int.=intergenic.

FIG. 2: Alignment of KU70 homologues. Sc=Saccharomyces cerevisiae, Hs=Homo sapiens and At=Arabidopsis thaliana. Perfect identity is depicted as black boxes, homology is depicted as grey boxes and dashes are used to optimize alignment.

FIG. 3: Alignment of LIG4 homologues. Sc=Saccharomyces cerevisiae, Hs=Homo sapiens and At=Arabidopsis thaliana. Perfect identity is depicted as black boxes, homology is depicted as grey boxes and dashes are used to optimize alignment.

FIG. 4: Alignment of MRE11 homologues. Sc=Saccharomyces cerevisiae, Hs=Homo sapiens and At=Arabidopsis thaliana. Perfect identity is depicted as black boxes, homology is depicted as grey boxes and dashes are used to optimize alignment.

FIG. 5: Alignment of RAD50 homologues. Sc=Saccharomyces cerevisiae, Hs=Homo sapiens and At=Arabidopsis thaliana. Perfect identity is depicted as black boxes, homology is depicted as grey boxes and dashes are used to optimize alignment.

FIG. 6: Alignment of XRCC4 homologues. Sc=Sac charomyces cerevisiae,Hs=Homo sapiens and At=Arabidopsis thaliana.

### DETAILED DESCRIPTION OF THE INVENTION

Experimental Part

Yeast Strains.

The yeast strains that were used are listed in Table 1. Yeast mutants isogenic to the haploid YPH250 strain were constructed using the one-step disruption method (Rothstein, 1991). A 1987 bp fragment from the YKU70 locus was amplified by PCR using the primers hdflp1 5′-GGGATTGCTTTAAGGTAG-3′ and hdflp2 5′-CAAATACCCTACCCTACC-3′. The PCR product was cloned into pT7Blue (Novagen) to obtain pT7BlueYKU70. An 1177 bp EcoRV/HindIII fragment from the YKU70 ORF was replaced by a 2033 bp HindIII/SmaI LEU2-containing fragment from pJJ283 (Jones and Prakash, 1990), to form pT7BlueYKU70::LEU2. In order to obtain YKU70 disruptants, Leu⁺ colonies were selected after transformation of YPH250 with a 2884 bp NdeI/SmaI fragment from pT7B1ueYKU70::LEU2. The Expand™ High Fidelity System (Boehringer Mannheim) was used according to the supplied protocol to amplify a 3285 bp fragment from the LIG4 locus with primers dnl4p1 5′-CGTAAGATTCGCCGAGTATAG-3′ and dnl4p2 5′-CGTTTCAAATGGGACCACAGC-3′. The PCR product was cloned into pGEMT (Promega), resulting in pGEMTLIG4. A 1326 bp BamHI/AhoI fragment from pJJ215 (Jones and Prakash, 1990) containing the HIS3 gene was inserted into the BamHI and XhoI sites of pIC20R, resulting in pIC20RHIS3. A 782 bp EcoRI fragment from the LIG4 ORF was replaced with a 1367 bp EcoRI HIS3-containing fragment from pIC20RHIS3 to construct pGEMTLIG4::HIS3. In order to obtain LIG4 disruptants, His⁺ colonies were selected after transformation of YPH250 with a 3854 bp NcoI/NotI fragment from pGEMTLIG4::HIS3. In order to obtain RAD50 disruptants, YPH250 was transformed with an EcoRI/Bg/II fragment from pNKY83, and Ura+colonies were selected (Alani et al., 1989). A rad50::hisG strain was obtained by selecting Ura⁺colonies on selective medium containing 5-FOA. Similarly, RAD51 disruptants were obtained after transformation of YPH250 with a RAD51::LEU2 XbaI/PstI fragment from pDG152 and selection of Leu⁺ colonies (Schiestl et al., 1994). The TRPI marker in pSM21 (Schild et al., 1983) was replaced with a BglII/XbaI LEU2-containing fragment from pJJ283 (Jones and Prakash, 1990). This resulted in pSM21LEU2. Leu⁺ RAD52 disruptant colonies were selected after transformation of YPH250 with the RAD52::LEU2 BamHI fragment from pSM21LEU2. Disruption constructs were transformed to YPH250 by the lithium acetate transformation method as described (Gietz et al., 1992; Schiestl et al., 1993). Disruption of YKU70, LIG4, RAD50, RAD51 and RAD52 was confirmed by PCR and Southern blot analysis.

TABLE 1
Yeast strains

| | | |
|---|---|---|
| Strain | Genotype | Reference |

| | | |
|---|---|---|
| YPH250 | MATa, ura3-52, lys2-801, | (Sikorski and |
| | ade2-101, trp1-Δ1, his3-Δ200, | Hieter, 1989) |
| | leu2-Δ1 | |
| YPH250rad51 | MATa, ura3-52, lys2-801, | This study |
| | ade2-101, trp1-Δ1, his3-Δ200, | |
| | leu2-Δ1, rad51::LEU2 | |
| YPH250rad52 | MATa, ura3-52, lys2-801, | This study |
| | ade2-101, trp1-Δ1, his3-Δ200, | |
| | leu2-Δ1, rad52::LEU2 | |
| YPH250yku70 | MATa, ura3-52, lys2-801, | This study |
| | ade2-101, trp1-Δ1, his3-Δ200, | |
| | leu2-Δ1, yku70::wLEU2 | |
| YPH250rad50 | MATa, ura3-52, lys2-801, | This study |
| | ade2-101, trp1-Δ1, his3-Δ200, | |
| | leu2-Δ1, rad50::hisG | |
| YPH250lig4 | MATa, ura3-52, lys2-801, | This study |
| | ade2-101, trp1-Δ1, his3-Δ200, | |
| | leu2-Δ1, lig4::HIS3 | |
| JKM115 | Δho, Δhml::ADE1, MATa, | (Moore and |
| | Δhmr::ADE1, ade1, | Haber, 1996) |
| | leu2-3, 112, lys5, trp1::hisG, | |
| | ura3-52 | |
| JKM129 | Δho, Δhml::ADE1, MATa, | (Moore and |
| | Δhmr::ADE1, ade1, | Haber, 1996) |
| | leu2-3, 112, lys5, trp1::hisG, | |
| | ura3-52, xrs2::LEU2 | |
| JKM138 | Δho, Δhml::ADE1, MATa, | (Moore and |
| | Δhmr::ADE1, ade1, | Haber, 1996) |
| | leu2-3, 112, lys5, trp1::hisG, | |
| | ura3-52, mre11::hisG | |
| YSL204 | Δho, HMLa, MATa, HMRa, | (Lee et al., 1999) |
| | ade1-100, leu2-3, 112, lys5, | |
| | trp1::hisG, ura3-52, | |
| | hisG′-URA3-hisG′, sir4::HIS3 | |

Construction of Binary Vectors.

To construct pSDM8000, a 1513 bp PvuII/EcoRV fragment carrying the KanMX marker was obtained from pFA6a (Wach et al., 1994) and was ligated into the unique HpaI site of pSDM14 (Offringa, 1992). pSDM8001 was made in three cloning steps. A 1476 bp BamHI/EcoRI fragment carrying the KanMX marker was obtained from pFA6a and ligated into BamHI- and EcoRI-digested pIC20H to form pIC20HkanMX. The KanMX marker was inserted between the PDA1 flanks by replacement of a 2610 bp BglII fragment from pUC4E1a10 (Steensma et al., 1990) with a 1465 Bg/II fragment from pIC2OHkanMX. A 3721 bp xhoI/KpnI fragment from this construct was inserted into the XhoI and KpnI sites of pSDM14. The binary vectors pSDM8000 and pSDM8001 were introduced into Agrobacterium tumefaciens LBA1119 by electroporation (den Dulk-Ras and Hooykaas, 1995).

Cocultivations/T-DNA Transfer Experiments.

Cocultivations were performed as described earlier with slight modifications (Bundock et al., 1995). Agrobacterium was grown overnight in LC medium. The mix of Agrobacterium and S. cerevisiae cells was incubated for nine days at 20° C. G418-resistant S. cerevisiae strains were selected at 30° C. on YPAD medium containing geneticin (200 μg/ml) (Life Technologies/Gibco BRL). Vectorette PCR.

Chromosomal DNA was isolated using Qiagen's Genomic Tips G/20 per manufacturer's protocol. 1-2 μg of Genomic DNA was digested with EcoRI, ClaI, PstI or HindIII and run on a 1% TBE-gel. Nonradioactive Southern blotting was performed. The membrane was hybridized with a digoxigenine-labeled kanMX probe to determine the size of T-DNA/genomic DNA fragments (EcoRI and Clal for RB-containing fragments and PstI and HindIII for LB-containing fragments). The kanMX probe, a 792 bp internal fragment of the KanMX marker, was made by PCR using primers kanmxpl 5′-AGACTCACGTTTCGAGGCC-3′ and kanmxp2 5′-TCACCGAGGCAGTTCCATAG-3′ and a Nonradioactive DNA Labeling and Detection kit (Boehringer Mannheim). The enzyme showing the smallest band on blot was used for Vectorette PCR in order to amplify the smallest junction sequence of T-DNA and genomic DNA. Vectorette PCR was performed as described (http://genomewww.stanford.edu/group/botlab/protocols/vectorette.html). The Expand™ High Fidelity System (Boehringer Mannheim) was used to amplify fragments larger than 2.5 kb, whereas sTaq DNA polymerase (SphaeroQ) was used for amplification of fragments smaller than 2.5 kb. Primers kanmxp3 5′-TCGCAGGTCTGCAGCGAGGAGC-3′ and kanmxp4 5′-TCGCCTCGACATCATCTGCCCAG-3′ were used to amplify RB/genomic DNA and LB/genomic DNA junction sequences, respectively.

T7 DNA Polymerase Sequencing.

Vectorette PCR products were cloned in pGEMTEasy (Promega) and sequenced using the T7 polymerase sequencing kit (Pharmacia) according to the manufacturer's protocol. In order to obtain sequences flanking the RB and LB, primers kanmxp5 5′-TCACATCATGCCCCTGAGCTGC-3′ and kanmxp4 were used, respectively.

### RESULTS

1. Binary Vectors for T-DNA Transfer to Yeast.

It was previously demonstrated that Agrobacterium tumefaciens is able to transfer its T-DNA not only to plants but also to another eukaryote, namely, the yeast Saccharomyces cerevisiae (Bundock et al., 1995). T-DNA carrying homology with the yeast genome was shown to become integrated by homologous recombination. T-DNA lacking any homology with the yeast genome was integrated randomly into the genome by IR, like in plants (Bundock et al., 1995; Bundock and Hooykaas, 1996). The T-DNA used in these experiments carried the S. cerevisiae URA3 gene for selection of Ura⁺ colonies after T-DNA transfer to the haploid yeast strain RSY12(URA3Δ). However, in this system, only yeast strains could be used in which the URA3 gene had been deleted to avoid homology between the incoming T-DNA and the S. cerevisiae genome.

We wanted to set up a system in which T-DNA transfer to any yeast strain could be studied. Therefore, two new binary vectors were constructed using the dominant marker kanMX (Wach et al., 1994), which confers resistance against geneticin (G418). The T-DNA of pSDM8000 carries only the KanMX marker. Since this KanMX marker consists of heterologous DNA, lacking any homology with the S. cerevisiae genome, we would expect this T-DNA to integrate by IR at a random position in the yeast genome. To be able to compare this with T-DNA integration by homologous recombination, pSDM8001 was constructed. The T-DNA of pSDM8001 carries the KanMX marker flanked by sequences from the S. cerevisiae PDA1 locus. The PDA1 sequences have been shown to mediate the integration of T-DNA by HR at the PDA1 locus on chromosome V (Bundock et al., 1995).

Cocultivations between Agrobacterium strains carrying pSDM8000 and pSDM8001, respectively, and the haploid yeast strains YPH250 and JKM115, respectively, were carried out as described in the experimental part. G418-resistant colonies were obtained at low frequencies for YPH250 (1.6×10⁻⁷) and JKM115 (1.2×10⁻⁵) after T-DNA transfer from pSDM8000 (Table 2). T-DNA transfer from pSDM8001-generated G418-resistant colonies at higher frequencies (2.4×10⁻⁵ for YPH250 and 1.8×10⁴ JKM115, Table 2). The ratio of homologous recombination versus illegitimate recombination is determined by comparing the frequencies of G418-resistant colonies obtained from cocultivations using either pSDM8001 or pSDM8000. This showed that a T-DNA from pSDM8001 was 150-fold more likely to integrate than a T-DNA from pSDM8000 in YPH250 (Table 2). A similar difference was previously seen using T-DNAs with the URA3 marker (Bundock and Hooykaas, 1996). In contrast, T-DNA from pSDM8001 was only 16-fold more likely to integrate than a T-DNA from pSDM8000 in JKM115. There was no significant difference in the frequency of T-DNA transfer to these two yeast strains as was determined by T-DNA transfer experiments in which a T-DNA that carried the KanMX marker and the yeast 2 micron replicon was employed. Therefore, the differences in the frequencies of T-DNA integration by HR and IR between the yeast strains YPH250 and JKM115, respectively, most likely contributed to differences in the capacities of their HR and IR recombination machineries.

We confirmed by PCR that the T-DNA from pSDM8001 became integrated at the PDA1 locus by homologous recombination (data not shown). In order to find out whether the T-DNA from pSDM8000 had integrated randomly by IR, yeast target sites for integration were determined from eight G418-resistant YPH250 colonies by Vectorette PCR (for detailed description see materials and methods). Chromosomal DNA was isolated and digested with a restriction enzyme that cuts within the T-DNA. A Vectorette was ligated to the digested DNA and a PCR was performed using a T-DNA-specific primer and a Vectorette-specific primer. The PCR product obtained was cloned into pGEMTEasy and sequenced using a T-DNA-specific primer. The position of the T-DNA insertion was determined by basic BLAST search of the yeast genome (http://www-genome.stanford.edu/SGD). We were thus able to map the position of the T-DNA insertions of all eight G418-resistant colonies analyzed. They were present at different positions spread out over the genome. Comparison of the T-DNA sequence and yeast target site sequences did not reveal any obvious homology. These data show that the T-DNA from pSDM8000 had integrated via an IR mechanism as expected.

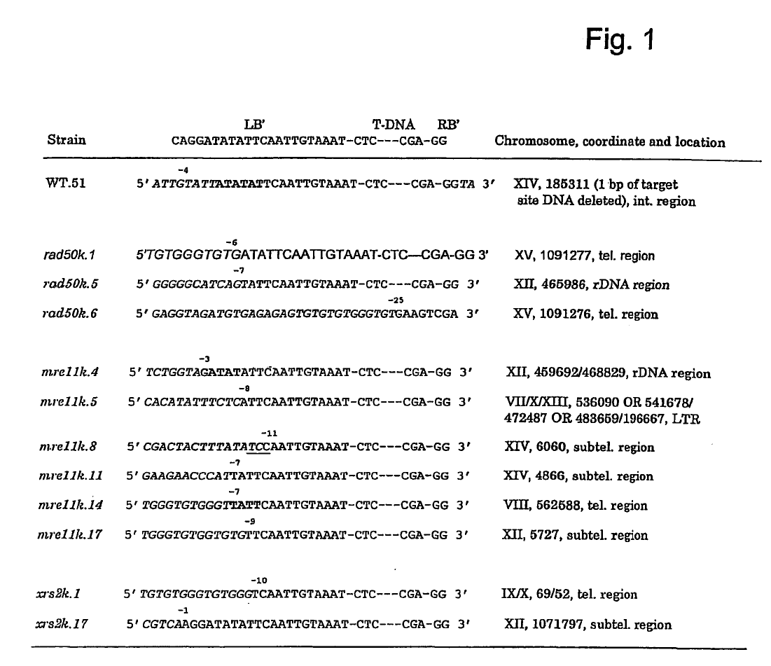

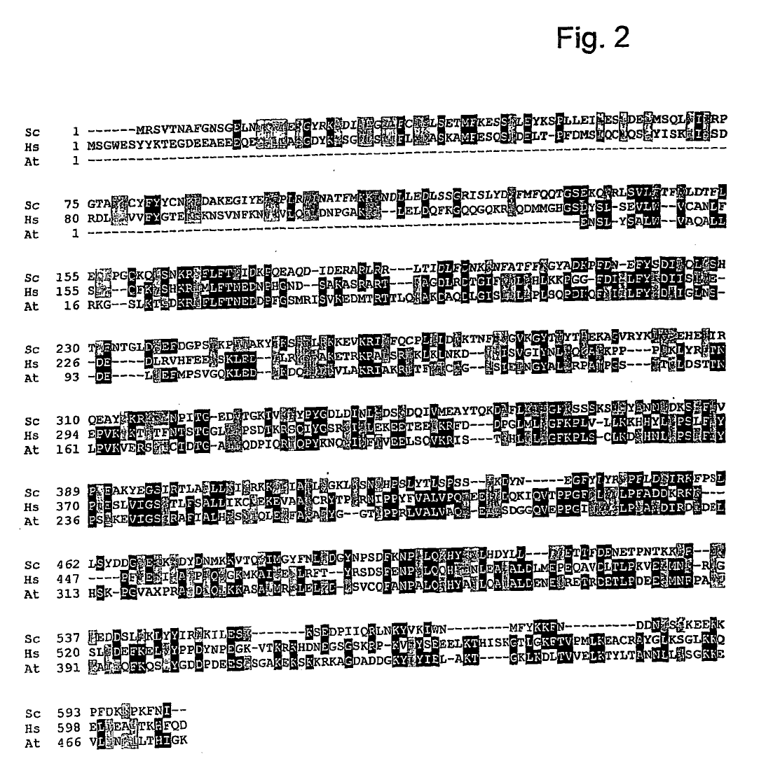

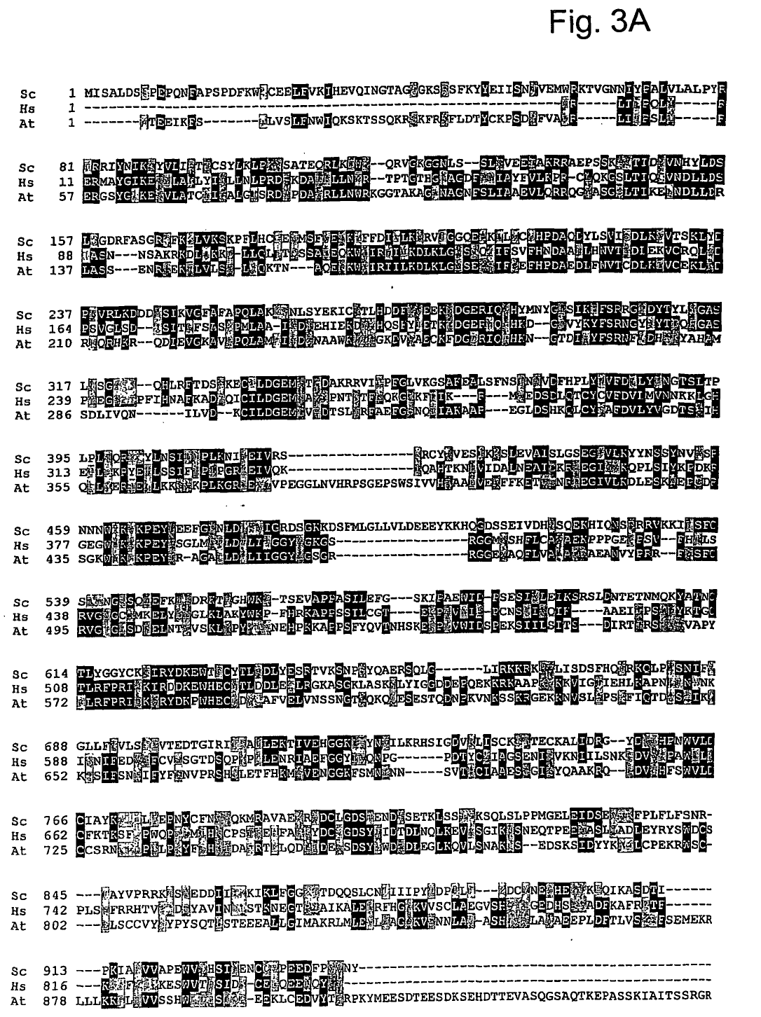

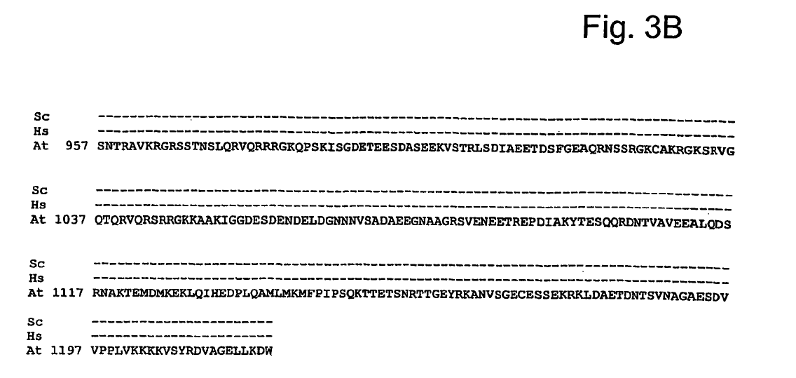

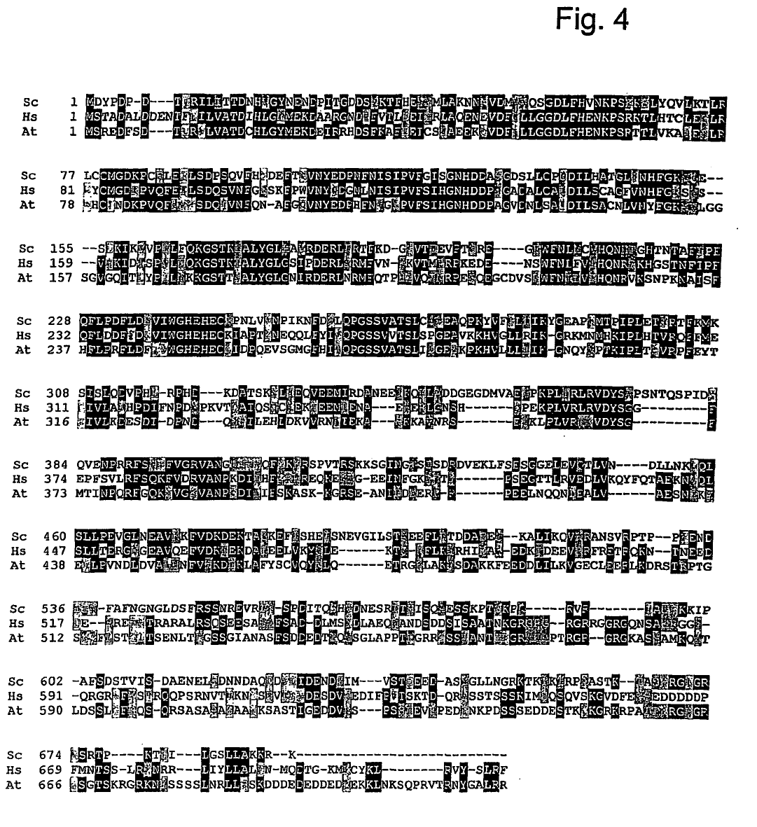

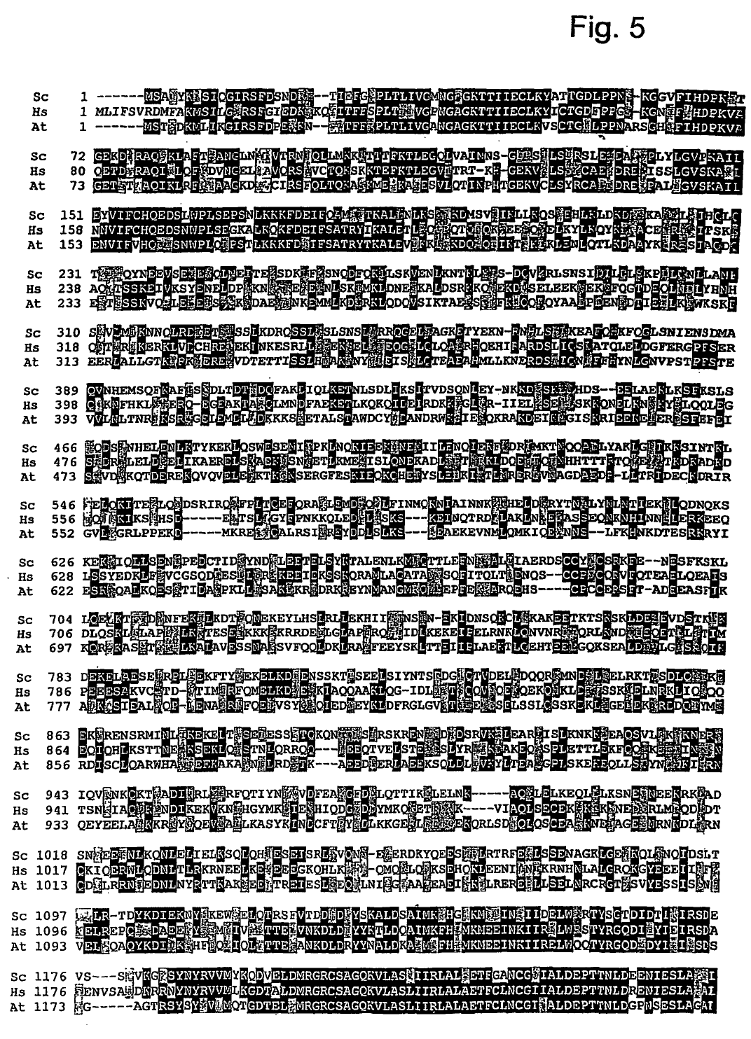

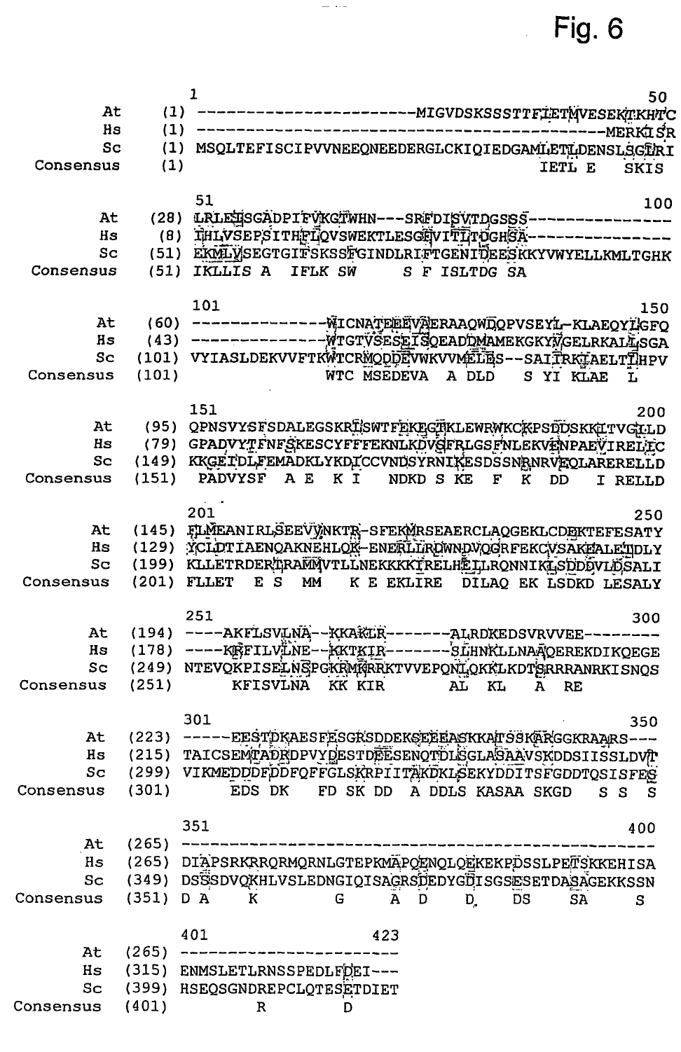

The following characteristics have previously been observed for T-DNAs integrated by IR: a) the 3′ end of the T-DNA is usually less conserved compared to the 5′ end, b) microhomology is sometimes present between the T-DNA ends and the target site, c) integration is often accompanied by small deletions of the target site DNA (Matsumoto et al., 1990; Gheysen et al., 1991; Mayerhofer et al., 1991; Bundock and Hooykaas, 1996). Similar characteristics were seen in the currently analyzed eight T-DNA insertions. In three strains, we observed microhomology of 2-6 bp between the LB and yeast target site (FIG. 1, WT.51 was taken as an example). In five strains, deletions of 1-5 bp of yeast target site DNA was found and we observed deletions, varying from 1-112 bp, of the 3′ end of the T-DNA in seven out of eight analyzed strains. In only one strain, the 3′ end appeared to be intact. The 5′ end of the T-DNA was conserved in almost all strains. In only two strains, we observed small deletions of 1 and 2 bp at the 5′ end of the T-DNA.

Thus, we can conclude that the T-DNA from pSDM8000 had integrated via the same IR mechanism described before.

TABLE 2
Frequencies of T-DNA integration by JR relative to integration by HR
in recombination defective yeast strains

| | | | | Absolute | Standardized |
|---|---|---|---|---|---|
| Strain | Genotype | Freq. of Ir^{a} | Freq. of HR | IR/HR ratio^{b} | IR/HR ratio^{c} |
| YPH250 | WT | 1.6 × 10⁻⁷ | 2.4 × 10⁻⁵ | 0.007 | 1 |
| YPH250 | rad51Δ | 1.4 × 10⁻⁷ | 1.5 × 10⁻⁶ | 0.09 | 14 |
| rad51 | | | | | |
| YPH250 | rad52Δ | 3.8 × 10⁻⁷ | 2.5 × 10⁻⁶ | 0.15 | 23 |
| rad52 | | | | | |
| YPH250 | yku70Δ | <3.2 × 10⁻⁹ | 3.3 × 10⁻⁵ | <0.0001 | <0.01 |
| yku 70 | | | | | |
| YPH250 | rad50Δ | 8.0 × 10⁻⁹ | 3.5 × 10⁻⁵ | 0.0002 | 0.03 |
| rad50 | | | | | |
| YPH250 | lig4Δ | 3.7 × 10⁻⁹ | 2.3 × 10⁻⁵ | 0.0002 | 0.02 |
| lig4 | | | | | |
| JKM115 | WT | 1.2 × 10⁻⁵ | 1.8 × 10⁻⁴ | 0.07 | 1 |
| JKM129 | xrs2Δ | 2.7 × 10⁻⁷ | 5.1 × 10⁻⁵ | 0.005 | 0.08 |
| JKM138 | mre11Δ | 2.9 × 10⁻⁷ | 7.5 × 10⁻⁵ | 0.004 | 0.06 |
| YSL204 | sir4Δ | 1.5 × 10⁻⁷ | 1.8 × 10⁻⁵ | 0.008 | 0.13 |

^{a}Averages of two or more independent experiments are shown. Frequencies are depicted as the number of G418-resistant colonies divided by the output number of yeast cells (cells/ml).
^{b}The frequency of T-DNA integration by IR (pSDM8000) divided by the frequency of T-DNA integration by HR (pSDM8001).
^{c}The ratio of IR/HR in the mutant strain divided by the ratio of IR/HR in the wild-type strain.

2. Host-specific Factors Involved in Random T-DNA Integration.

The observation that the T-DNA from pSDM8000 integrates by IR into the yeast genome allowed us to use this system to study the effect of host factors on the process of integration. Many proteins involved in various forms of DNA recombination have been identified in yeast. In order to determine the roles of a representative set of these enzymes in T-DNA integration, we compared T-DNA transfer and integration in wild-type yeasts with that of strains carrying disruptions of the genes encoding several recombination proteins. The RAD51, RAD52, KU70, RAD50 and LIG4 genes were deleted from YPH250 using the one step disruption method. Yeast strains carrying deletions in MRE11, XRS2 and SIR4 in the JKM115 background were kindly provided by Dr. J. Haber. The results of cocultivations with these yeast strains are shown in Table 2.

In rad51and rad52 mutants, which are impaired in homologous recombination, the frequency of T-DNA integration by HR was sixteen- and nine-fold lower, respectively, than observed for the wild-type (Table 2). This showed that RAD51 and RAD52 play a role in T-DNA integration by homologous recombination. In the IR defective ku70, rad50, lig4, mre11, xrs2 and sir4 mutants, the frequency of T-DNA integration by HR did not differ significantly from that observed for wild-type (Table 2). This showed that these genes do not play a role in T-DNA integration by homologous recombination.

The frequency of T-DNA integration by IR in a rad51 mutant did not differ significantly from that observed for wild-type, whereas in a rad52 mutant, the frequency was about two-fold higher (Table 2). This showed that RAD51 and RADS2 are not involved in T-DNA integration by IR. The product of the RAD52 gene may compete with IR-enzymes for the T-DNA and thereby inhibits integration by IR to some extent. Strikingly, in rad50, mre11, xrs2, lig4 and sir4 mutants, the frequency of T-DNA integration by IR was reduced dramatically: 20- to more than 40-fold (Table 2). T-DNA integration by IR seemed to be completely abolished in the ku70 mutant. We did not obtain any G418-resistant colonies from several cocultivation experiments. This strongly suggests that KU70 plays an important role in random T-DNA integration in yeast.

Since T-DNA integration by HR is normal in these mutants, these results clearly show that the yeast genes KU70, RAD50, MRE11, XRS2, LIG4 and SIR4 are involved in T-DNA integration by illegitimate recombination.

3. Chromosomal Distribution of Integrated T-DNA Copies in IR-defective S. cerevisiae.

From several cocultivation experiments with the rad50, mre11, xrs2, lig4 and sir4 mutants, we obtained a small number of G418-resistant colonies. The T-DNA structure was determined for a number of these lines. To this end, chromosomal DNA was isolated from these G418-resistant colonies and subjected to vectorette PCR to amplify junction sequences of genomic DNA and T-DNA. PCR products were cloned and sequenced. The yeast sequences linked to the T-DNA were used in a BLAST search at http://www-genome.stanford.edu/SGD to determine the T-DNA integration sites.

Strikingly, analysis of LB/genomic DNA junctions revealed that in two out of three rad50, four out of six mre11 and two xrs2 strains analyzed, T-DNAs had integrated in telomeres or subtelomeric regions (rad50k.1, rad50k.6, mre11k.8, mre11k.11, mre11k.14, mre11k17, xrs2k.1 and xrs2k. 17; Table 3 and FIG. 1). S. cerevisiae telomeres generally consist of one or more copies of the Y′ element followed by telomerase-generated C(1-3)A/TG(1-3) repeats (Zakian, 1996). In two rad50strains, two mre11 strains and one xrs2 strain, the LB was found to be fused to this typical telomerase-generated C(1-3)A/TG(1-3) repeat (rad50k.1, rad50k. 6, mre11k. 14, mre11k. 17 and xrs2k. 1; FIG. 1). Besides, we also found one T-DNA insertion in a Ty LTR element in the mre11 mutant and two insertions in the rDNA region, present in chromosome XII, in the mre11 and rad50mutants (mre11k.5, mre11k.4 and rad50k.5, respectively; Table 3 and FIG. 1).

The 3′ end of the T-DNA was truncated in all strains. Deletions of 3-11 bp of the 3′ end of the T-DNA were observed (FIG. 1). Microhomology between the 3′ end of the T-DNA and yeast target site was only found in two lines (5 bp in mre11k.4 and 4 bp in mre11k 14; FIG. 1). For the T-DNA copies present at the yeast telomeres, the RB/genomic DNA junction sequences could not be obtained from these strains using vectorette PCR. This was only possible for the rad50and mre11 strains carrying the T-DNA in the rDNA region on chromosome XII. In both strains, the RB was intact and no homology between the 5′ end of the T-DNA and the yeast target site was found (data not shown in FIG. 1).

Previously, target sites for T-DNA integration in the genome of S. cerevisiae strain RSY12 were determined (Bundock and Hooykaas, 1996; Bundock, 1999). In four out of 44 strains analyzed, T-DNA copies were integrated in rDNA, Ty LTR elements (in two strains) and a subtelomerically located Y′ element, respectively. In addition, we determined the position of T-DNA integration in ten S. cerevisiae YPH250 strains. We did not find any T-DNA insertions in rDNA, LTR elements or subtelomeric/telomeric regions amongst these ten. Pooling all insertions analyzed in wild-type (54), in two out of 54 strains analyzed (4%), insertions were found in a Ty LTR element and in two other strains in the rDNA repeat (2%) and a subtelomeric region (2%), respectively. In contrast, we report here that T-DNA in yeast strains mutated in RAD50, MRE11 or XRS2 T-DNA integrates preferentially in (sub)telomeric regions (eight out of eleven lines: ˜73%) of rad50, mre11 and xrs2 mutants (Table 3). From the remaining strains, two T-DNAs were present in rDNA and one in a Ty LTR element, respectively. Apparently, the rDNA repeat is also a preferred integration site in these mutants (˜18% vs. ˜2% in the wild-type).

Telomeres consist of a large array of telomerase-generated C(1-3)A/TG(1-3) repeats (˜350 bp). In the subtelomeric regions, two common classes of Y′ elements, 6.7 and 5.2 kb, can be found (in most strains, chromosome I does not contain Y′) (Zakian and Blanton, 1988), making the average size of these regions ˜6.0 kb. Thus, the yeast genome contains (16×2×0.35)+(15×2×6.0)=191 kb of subtelomeric/telomeric sequences. The yeast genome is 12,052 kb in size, which means that only 1.6% of the genome consists of subtelomeric/telomeric sequences. In accordance with this, we observed in only 2% of the wild-type strains T-DNA copies inserted in a subtelomeric region, which we would expect on the basis of random T-DNA integration. In contrast, in the rad50, mre11 and xrs2 mutants, 73% of the T-DNA insertions were found in the (sub)telomeric region.

Analysis of seven lines revealed that in the sir4 mutant T-DNA was integrated randomly into the yeast genome. So, although SIR4 has an effect on the efficiency of T-DNA integration by IR, the pattern of T-DNA distribution in the transformants seems similar as in the wild-type strain. In the sir4 mutant T-DNA, integration by IR was characterized by truncation of the 3′ end of the T-DNA, deletions at the target site and microhomology between the LB and the target site (data not shown); this was observed for T-DNA integration by IR in the wild-type.

These results clearly show that in the rad50, mre11 and xrs2 mutants, the T-DNA, if integrated at all, becomes preferentially inserted in telomeres or subtelomeric regions and that the genomic distribution of integrated T-DNAs is altered when compared to wild-type. However, disruption of SIR4 did affect the efficiency of T-DNA integration by IR but not the characteristics of this process.

TABLE 3
genomic distribution of T-DNA integrated by IR
in rad50, mre11 and xrs2 mutants in comparison
with the wild-type after T-DNA transfer from pSDM8000

| | (Sub)Telomeric | | | |
|---|---|---|---|---|
| Yeast strain | region | LTR | rDNA | Elsewhere |
| rad50 mutant | 2 | 0 | 1 | 0 |
| mre11 mutant | 4 | 1 | 1 | 0 |
| xrs2 mutant | 2 | 0 | 0 | 0 |
| wild-type | 1 | 2 | 1 | 50 |

### REFERENCE LIST

Alani, E., Subbiah, S., and Kleckner, N. 1989. The yeast RAD50 gene encodes a predicted 153-kD protein containing a purine nucleotide-binding domain and two large heptad-repeat regions. Genetics 122:47-57.

Bechtold, N., Ellis, J. and Pelletier, G. 1993. In planta Agrobacterium mediated gene transfer by infiltration of adult Arabidopsis thaliana plants. C. R. Acad. Sci. Paris Life Sci. 316, 1194-1199.

Bundock, P. 1999. Agrobacterium tumefaciens-mediated transformation of yeast and fungi. Thesis Leiden University.

Bundock, P., den Dulk-Ras, A., Beijersbergen, A., and Hooykaas, P. J. J. 1995. Trans-kingdom T-DNA transfer from Agrobacterium tumefaciens to Saccharomyces cerevisiae. EMBO. J. 14:3206-3214.

Bundock, P. and Hooykaas, P. J. J. 1996. Integration of Agrobacterium tumefaciens T-DNA in the Saccharomyces cerevisiae genome by illegitimate recombination. Proc. Natl. Acad. Sci. U.S.A 93:15272-15275.

Capecchi, M. R. 1980. High efficiency transformation by direct microinjection of DNA into cultured mammalian cells. Cell. 22, 479-488. de Groot, M. J. A., Bundock, P., Hooykaas, P. J. J., and Beijersbergen, A. G. M. 1998. Agrobacterium tumefaciens-mediated transformation of filamentous fungi. Nat. Biotech. 16:839-842.

den Dulk-Ras, A. and Hooykaas, P. J. J. 1995. Electroporation of Agrobacterium tumefaciens. Methods Mol. Biol. 55:63-72.

D'Halluin, K., Bonne, E., Bossut, M., Beuckeleer, M. D., and Leemans, J. 1992. Transgenic maize plants by tissue electroporation. Plant Cell. 4, 1495-1505.

Dunwell, J. M. 1999. Transformation of maize using silicon carbide whiskers. Meth. Mol. Biol. 111:371-382.

Gheysen, G., Villarroel, R., and Van Montagu, M. 1991. Illegitimate recombination in plants: a model for T-DNA integration. Genes Dev. 5:287-297.

Gietz, D., St. Jean, A., Woods, R. A., and Schiestl, R. H. 1992. Improved method for high efficiency transformation of intact yeast cells. Nucleic Acids Res. 20:1425.

Gietz, R. D. and Woods, R. A. 2001. Genetic transformation of yeast. Biotechniques 30, 816-820.

Hanover, J. A. 1992. The nuclear pore: at the crossroads. FASEB J. 6, 2288-2295.

Hansen, G. and Chilton, M. D. 1999. Lessons in gene transfer to plants by a gifted microbe. Curr. Top. Microbiol. Immunol. 240:21-57.

Hartung, F. and Puchta, H. 1999. Isolation of the complete cDNA of the MREI I homologue of Arabidopsis (Accession No. AJ243822) indicates conservation of DNA recombination mechanisms between plants and other eukaryotes. Plant Phys. 121:312-312.

Hooper, M. L. 1992. Embryonal stem cells: introducing planned changes into the animal germline, in Modern genetics Vol. 1, 1st ed., Evas, H. J., Chur: Harwood Academic Publishers.

Hooykaas, P. J. J. and Beijersbergen, A. G. M. 1994. The virulence system of Agrobacterium tumefaciens. Annu. Rev. of Phytopathol. 32:157-179.

Jones, J. S. and Prakash, L. 1990. Yeast Saccharomyces cerevisiae selectable markers in pUC18 polylinkers. Yeast 6:363-366.

Junop M. S., Modesti M., Guarne, A., Ghirlando R., Gellert M., Yang W. 2000. Crystal structure of the Xrcc4 DNA repair protein and implications for endjoining. EMBO J. 19, 5962-5970.

Kaeppler, H. E., Gu, W., Somers, D. A., Rines, H. W., Cockburn, A. F. 1990. Silicon carbide fiber-mediated DNA delivery into plant cells. Plant Cell Rep. 9, 415-418.

Kegel A., Sjostrand J. O., Astrom S. U. 2001. Nejlp, a cell type-specific regulator of nonhomologous endjoining in yeast. Curr. Biol. 11:1611-1617.

Klein, T. M., Arentzen, R., Lewis, P. A., and Fitzpatrick-McElligott, S. 1992. Transformation of microbes, plants and animals by particle bombardment. Biotechn. 10, 286-291.

Kunik, T., Tzfira, T., Kapulnik, Y., Gafni, Y., Dingwall, C., and Citovsky, V. 2001. Genetic transformation of HeLa cells by Agrobacterium. Proc. Natl. Acad. Sci. U.S.A. 98, 1871-1876.

Lee, S. E., Paques, F., Sylvan, J., and Haber, J. E. 1999. Role of yeast SIR genes and mating type in directing DNA double-strand breaks to homologous and nonhomologous repair paths. Curr. Biol. 9:767-770.

Lin, C. H., xiao, L., Hou, B. H., Ha, S. B., and Saunders, J. A. 1997. Optimization of electroporation conditions for expression of GUS activity in electroporated protoplasts and intact plant cells. Plant Physiology and Biochemistry, 35, 959-968.

Marangoni, E., Foray, N., O'Driscoll, M., Douc-Rasy, S., Bernier, J., Bourhis, J., Jeggo, P. 2000. A KU80 fragment with dominant negative activity imparts a radiosensitive phenotype to CHO-KI cells. Nucl. Acids. Res. 28, 4778-4782.

Matsumoto, S., Ito, Y., Hosoi, T., Takahashi, Y., and Machida, Y. 1990. Integration of Agrobacterium T-DNA into a tobacco chromosome: possible involvement of DNA homology between T-DNA and plant DNA. Mol. Gen. Genet. 224:309-316.

Mayerhofer, R., Koncz-Kalman, Z., Nawrath, C., Bakkeren, G., Crameri, A., Angelis, K., Redei, G. P., Schell, J., Hohn, B., and Koncz, C. 1991. T-DNA integration: a mode of illegitimate recombination in plants. EMBO J. 10:687-704.

Milne, G. T., Jin, S., Shannon, K. B., Weaver, D. T. 1996. Mutations in two Ku homologs define a DNA end-joining repair pathway in Saccharomyces cerevisiae. Mol. Cell. Biol. 8, 4189-4198.

Moore, J. K. and Haber, J. E. 1996. Cell cycle and genetic requirements of two pathways of nonhomologous end-joining repair of double-strand breaks in Saccharomyces cerevisiae. Mol. Cell. Biol. 16:2164-2173.

Offringa, R. 1992. Gene targeting in plants using the Agrobacterium vector system. Thesis Leiden University.

Offringa, R., de Groot, M. J., Haagsman, H. J., Does, M. P., van den Elzen, P. J., and Hooykaas, P. J. J. 1990. Extrachromosomal homologous recombination and gene targeting in plant cells after Agrobacterium mediated transformation. EMBO J. 9:3077-3084.

Ozeki, K., Kyoya, F., Hizume, K., Kanda, A., Hamachi, M. and Nunokawa, Y. 1994. Transformation of intact Aspergillus niger by electroporation. Biosci. Biotechnol. Biochem. 12, 2224-2227.

Paull, T. T. and Gellert, M. 1999. Nbsl potentiates ATP-driven DNA unwinding and endonuclease cleavage by the Mre11/Rad50 complex. Genes Dev. 13, 1276-1288.

Picard, D., Salser, S. J., Yamamoto, K. R. 1988. A movable and regulatable inactivation finction within the steroid binding domain of the glucocorticoid receptor. Cell 54, 1073-1080.

Raikhel, N. 1992. Nuclear targeting in plants. Plant Physiol. 100, 1627-1632.

Rothstein, R. 1991. Targeting, disruption, replacement, and allele rescue: integrative DNA transformation in yeast. Methods Enzymol. 194:281-301.

Schiestl, R. H., Manivasakam, P., Woods R. A. and Gietz, R. D. 1993. Introducing DNA into yeast by transformation. Methods: A companion to methods in enzymology 5:79-85.

Schiestl, R. H., Zhu, J., and Petes, T. D. 1994. Effect of mutations in genes affecting homologous recombination on restriction enzyme-mediated and illegitimate recombination in Saccharomyces cerevisiae. Mol. Cell. Biol. 14:4493-4500.

Schild, D., Calderon, I. L., Contopoulou, C. R. and Mortimer, R. K. 1983. Cloning of yeast recombination repair genes and evidence that several are nonessential genes, p. 417-427. In: E. C. Friedberg and B. A. Bridges (eds.), Cellular responses to DNA damage. Alan R. Liss Inc., New York.

Schnorf, M., Neuhaus-Url, G., Galli, A., Iida, S., and Potrykus, I. 1991. An improved approach for transformation of plant cells by microinjection: molecular and genetic analysis. Transgenic Res. 1, 23-30.

Sikorski, R. S. and Hieter, P. 1989. A system of shuttle vectors and yeast host strains designed for efficient manipulation of DNA in Saccharomyces cerevisiae. Genetics 122:19-27.

Steensma, H. Y., Holterman, L., Dekker, I., van Sluis, C. A., and Wenzel, T. J. 1990. Molecular cloning of the gene for the El alpha subunit of the pyruvate dehydrogenase complex from Saccharomyces cerevisiae. Eur. J. Biochem. 191:769-774.

Taccioli, G. E., Gottlieb, T. M., Blunt, T., Priestley, A., Demengeot, J., Mizuta, R., Lehmann, A. R., Alt, F. W., Jackson, S. P., Jeggo, P.A. 1994. Ku8O: product of the XRCC5 gene and its role in DNA repair and V(D)J recombination. Science 265, 1442-1445.

Templeton, N. S., Roberts, D. D. and Safer, B. 1997. Efficient gene targeting in mouse embryonic stem cells. Gene Ther. 4, 700-709.

Teo, S. H. and Jackson, S. P. 2000. Lif1p targets the DNA ligase Lig4p to sites of DNA double-strand breaks. Curr. Biol. 10, 165-168.

Valencia, M., Bentele, M., Vaze, M. B., Hermann, G., Kraus, E., Lee, S. E., Schar, P., Haber, J. E. 2001. Nature 414, 666-669.

Vergunst, A. C., De Waal, E. C., and Hooykaas, P. J. J. 1998. Root transformation by Agrobacterium tumefaciens. In Arabidopsis protocols. J. Martinez-Zapater and J. Salinas, eds. (Totowa, N. J.: Humana Press, Inc.).

Vergunst, A. C., Schrammeijer, B., den Dulk-Ras, A., de Vlaam, C. M. T., Regensburg-Tuïnk, T. J. G., and Hooykaas, P. J. J. 2000. VirB/D4 dependent protein translocation from Agrobacterium into plant cells. Science: in press. Wach, A., Brachat, A., Pohlmann, R., and Philippsen, P. 1994. New heterologous modules for classical or PCR-based gene disruptions in Saccharomyces cerevisiae. Yeast 10:1793-1808.

PCT International Publication No. WO 00/68404, Pioneer Hi-bred.

PCT International Publication No. WO 00/127 16, Pioneer Hi-bred.

Zakian, V. A. 1996. Structure, function, and replication of Saccharomyces cerevisiae telomeres. Annu. Rev. Genet. 30:141-172.

Zakian, V. A. and Blanton, H. M. 1988. Distribution of telomere-associated sequences on natural chromosomes in Saccharomyces cerevisiae. Mol. Cell. Biol. 8:2257-2260.

Zrenner, R., Willmitzer, L., and Sonnewald, U. 1993. Analysis of the expression of potato uridinediphosphate-glucose pyrophosphorylase and its inhibition by antisense RNA. Planta, 190:247-252.

What is claimed is:

1. A method of directing integration of a nucleic acid of interest to a predetermined site, wherein said nucleic acid has homology at or around said predetermined site, in a eukaryote with a preference for nonhomologous recombination, said method comprising:
steering an integration pathway towards homologous recombination.

2. The method of directing nucleic acid integration according to claim 1, further comprising: providing a mutant of a component involved in nonhomologous recombination.

3. The method of directing nucleic acid integration according to claim 1 or 2, further comprising: inhibiting a component involved in nonhomologous recombination.

4. The method according to claim 2 or 3 wherein said component involved in nonhomologous recombination comprises ku70, rad50, mre11, xrs2, lig4 or sir4.

5. The method of directing integration of a nucleic acid of interest to a predetermined site according to any one of claims 1 to 3, wherein said nucleic acid of interest is essentially replacing a sequence within said eukaryote.

6. The method of directing integration of a nucleic acid of interest to a predetermined site according to claim 5, wherein said component involved in nonhomologous recombination comprises rad50 or xrs2.

7. A method of directing integration of a nucleic acid of interest to a subtelomeric region, a telomeric region, or a subtelomeric region and telomeric region in a eukaryote with a preference for nonhomologous recombination by providing a mutant of a component involved in nonhomologous recombination.

8. A method of directing integration of a nucleic acid of interest to a subtelomeric region, a telomeric region, or a subtelomeric region and telomeric region in a eukaryote with a preference for nonhomologous recombination, comprising inhibiting a component involved in nonhomologous recombination.

9. The method of directing integration according to claim 7 or 8 wherein said component involved in nonhomologous recombination comprises rad50, mre1 or xrs2.

10. The method according to any one of claims 1 to 9 wherein said eukaryote is selected from the group consisting of yeast, fungus, and an animal.

11. The method according to any one of claims 1 to 10, wherein said nucleic acid of interest is delivered to a cell of said eukaryote by Agrobacterium.

12. The method according to any one of claims 1-11 comprising transiently inhibiting integration via nonhomologous recombination.

13. The method according claim 12 wherein said transiently inhibiting is provided by an Agrobacterium Vir-fusion protein capable of inhibiting a component involved in nonhomologous recombination.

14. The method of directing integration according to claim 13 wherein said Agrobacterium Vir-fusion protein comprises VirF or VirE2.

15. The method according to claim 13 or 14 wherein said component involved in nonhomologous recombination comprises ku70, rad50, mre11, xrs2, lig4 or sir4.

16. The method according to any one of the foregoing claims wherein said nucleic acid of interest comprises an inactive gene to replace an active gene.

17. The method according to any one of claims 1-14, wherein said nucleic acid of interest comprises an active gene to replace an inactive gene.

18. The method according to any one of claims 1-14, wherein said nucleic acid of interest encodes a therapeutic proteinaceous substance.

19. The method according to any one of claims 1-14, wherein said nucleic acid of interest encodes a substance conferring resistance for an antibiotic substance to a cell.

20. The method according to any one of claims 1-14, wherein said nucleic acid of interest confers a desired property to said eukaryote.

21. The method according to any one of the foregoing claims wherein said nucleic acid of interest is part of a gene delivery vehicle.

22. Use of a method according to any one of claims 1 to 20 for improvement of gene-targeting efficiency.
